SEQUENCE LISTING (21 sequences)

SEQ ID NO: 1 - protein, 217 aa, Escherichia coli
MDIISVALKRHSTKAFDASKKLTPEQAEQIKTLLQYSPSSTNSQPWHFIVASTEEGKARVAKSAAGNYVFNERKMLDASHVVVFCAKTAMDDVWLKLVVDQEDADGRFATPEAKAANDKGRKFFADMHRKDLHDDAEWMAKQVYLNVGNFLLGVAALGLDAVPIEGFDAAILDAEFGLKEKGYTSLVVVPVGHHSVEDFNATLPKSRLPQNITLTEV

SEQ ID NO: 2 - DNA, 20 bp, Artificial Sequence
  PCR primer
gacaattaatcatcggctcg

SEQ ID NO: 3 - DNA, 23 bp, Artificial Sequence
  PCR primer
gcggataacaatttcacacagga

SEQ ID NO: 4 - DNA, 20 bp, Artificial Sequence
  PCR primer
cagagcattagcgcaaggtg

SEQ ID NO: 5 - DNA, 24 bp, Artificial Sequence
  PCR primer
cccagccgtggcattttattgttg

SEQ ID NO: 6 - DNA, 50 bp, Artificial Sequence
  PCR primer for site-specific random mutagenesis
caacaataaaatgccacggctgggagttggtnnnggatgggctgtattgc

SEQ ID NO: 7 - DNA, 50 bp, Artificial Sequence
  PCR primer for site-specific random mutagenesis
caacaataaaatgccacggctgggagttnnngctggatgggctgtattgc

SEQ ID NO: 8 - DNA, 24 bp, Artificial Sequence
  PCR primer
gagcgtaaaatgcttgatgcctcg

SEQ ID NO: 9 - DNA, 50 bp, Artificial Sequence
  PCR primer for site-specific random mutagenesis
cgaggcatcaagcattttacgctcgttgaacacnnnattaccggcagcgg

SEQ ID NO: 10 - DNA, 45 bp, Artificial Sequence
  PCR primer for site-specific random mutagenesis
cgaggcatcaagcattttacgctcnnngaacacgtaattaccggc

SEQ ID NO: 11 - DNA, 25 bp, Artificial Sequence
  PCR primer
gctgatatgcaccgtaaagatctgc

SEQ ID NO: 12 - DNA, 51 bp, Artificial Sequence
  PCR primer for site-specific random mutagenesis
gcagatctttacggtgcatatcagcgaagaacttgcgnnntttatcgttcg

SEQ ID NO: 13 - DNA, 37 bp, Artificial Sequence
  PCR primer for site-specific random mutagenesis
gcagatctttacggtgcatatcagcnnngaacttgcg

SEQ ID NO: 14 - DNA, 20 bp, Artificial Sequence
  PCR primer
gcttcagccagacatcgtcc

SEQ ID NO: 15 - DNA, 45 bp, Artificial Sequence
  PCR primer for site-specific random mutagenesis
cgaggcatcaagcattttacgctcgttnnncacgtaattaccggc

SEQ ID NO: 16 - DNA, 42 bp, Artificial sequence
  PCR primer
gcacgctagcaagcttccaccatggatatcatttctgtcgcc

SEQ ID NO: 17 - DNA, 41 bp, Artificial sequence
  PCR primer
gcacaagcttgctagctcattacacttcggttaaggtgatg

SEQ ID NO: 18 - DNA, 15 bp, Artificial sequence
  Kozak sequence
aagcttccaccatgg

SEQ ID NO: 19 - DNA, 20 bp, Artificial sequence
  Kozak sequence
aagcttgccgccagccatgg

SEQ ID NO: 20 - DNA, 50 bp, Artificial sequence
  PCR primer
agtccaagcttgccgccagccatggatatcatttctgtcgccttaaagcg

SEQ ID NO: 21 - DNA, 37 bp, Artificial sequence
  PCR primer
tgaggatccttacacttcggttaaggtgatgttttgc

(19) United States
(12) Patent Application Publication
(10) Pub. No.: US 2005/0013808 A1
(43) Pub. Date: Jan. 20, 2005

(54) Nitroreductase enzymes

(76) Inventors: Jane Isabel Grove, Birmingham (GB); Peter Francis Searle, Birmingham (GB); Andrew Lee Lovering, Birmingham (GB)

Correspondence Address: COZEN O'CONNOR, P.C., 1900 MARKET STREET, PHILADELPHIA, PA, 19103, US

(21) Appl. No.: 10/487,569

(22) PCT Filed: Aug. 21, 2002
(86) PCT No.: PCT/GB02/03833

Related U.S. Application Data
(60) Provisional application No. 60/326,846, filed on Oct. 3, 2001.

(30) Foreign Application Priority Data
Aug. 21, 2001 (GB) 0120294.4
Sep. 6, 2001 (GB) 0121662.1

(51) Int. Cl. A61K 38/44; C12Q 1/68; C07H 21/04; C12N 9/06

(52) U.S. Cl. 424/94.4; 435/6; 435/69.1; 435/191; 435/320.1; 536/23.2; 435/252.33

(57) ABSTRACT

Improved nitroreductase enzymes, particularly for use as prodrug converting enzymes are provided. In particular, single and double mutants of the E. coli NFSB nitroreductase, having improved properties for the activation of the prodrug CB 1954 for use in gene therapy are disclosed.

### BACKGROUND TO THE INVENTION

The present invention relates to mutated nitroreductase enzymes and the DNA encoding them, and their use in the conversion of prodrugs for the treatment of cancer.

One approach to treating cancer is to introduce a gene into the tumour cells that encodes an enzyme capable of converting a prodrug of relatively low toxicity into a potent cytotoxic drug. Systemic administration of the prodrug is then tolerated since it is only converted into the toxic derivative locally, in the tumour, by cells expressing the prodrug-converting enzyme. This approach is known as gene-directed enzyme prodrug therapy (GDEPT), or when the gene is delivered by means of a recombinant viral vector, virus-directed prodrug therapy (VDEPT) (McNeish et al, 1997).

An example of an enzyme/prodrug system is nitroreductase and the aziridinyl prodrug CB1954 (5-(aziridin-1-yl)-2,4-dinitrobenzamide) (Knox et al 1988). Following the observation that the Walker rat carcinoma cell line was particularly sensitive to CB1954, it was shown that this was due to the expression of the rat nitroreductase DT diaphorase. However, since CB 1954 is a poor substrate for the human form of this enzyme, human tumour cells are far less sensitive to CB1954. GDEPT was conceived as a way of introducing a suitable nitroreductase, preferably with greater activity against CB1954, in order to sensitise targeted cells. The Escherichia coli nitroreductase (EC1.6.99.7, alternatively known as the oxygen-insensitive NAD(P)H nitroreductase or dihydropteridine reductase, and often abbreviated to NTR) encoded by the NFSB gene (alternatively known as NFNB, NFSI, or DPRA) has been widely used for this purpose (Reviewed in Grove et al, 1999). The NFSB-encoded nitroreductase (NTR) is a homodimer that binds two flavin mononucleotide (FMN) cofactor molecules. Using NADH or NADPH as an electron donor, and bound FMN as a reduced intermediate, NTR reduces one or other of the two nitro-groups of CB 1954 to give either the highly toxic 4-hydroxylamine derivative or the relatively non-toxic 2-hydroxylamine. Within cells, 5-(aziridin-1-yl)-4-hydroxylamino-2-nitrobenzamide, probably via a further toxic metabolite, becomes very genotoxic (Knox et al, 1991). The exact nature of the lesion caused is unclear, but is unlike that caused by other agents. A particularly high rate of inter-strand cross-linking occurs and the lesions seem to be poorly repaired, with the result that CB 1954 is an exceptionally affective anti-tumour agent (Friedlos et al, 1992).

The structure of the NFSB NTR has been analysed by X-ray crystallography (Parkinson et al 2000, Lovering et al, 2001). Each monomer consists of 217 amino acids forming a four-stranded beta sheet (a fifth parallel strand is contributed by the other subunit) and ten α helices (A-K) and comprises a large hydrophobic core (residues 2-91 and 131-217), a two helix domain (E and F, residues 92-130) that protrudes from the core region, and an extensive dimer interface formed by parts of helices A, B, G, J and K. (NB: the domain assignments are from Lovering et al, and differ slightly from the earlier structure solved by Parkinson et al). Residues in what Parkinson et al designated as Helix G (residues 113-131) have been identified as being in or near the active site and are important in determining substrate specificity. Lovering et al assigns residues 110-131 to helix F and 135-157 to helix G. However, both papers agree that residues in this region form part of the opening to the substrate- and cofactor-binding pocket and that phenylalanine 124 is particularly important.

The NFSB NTR has sequence homology to a number of other enzymes, in particular FRase I, a flavin reductase enzyme from Vibrio fischeri (Zenno et al 1996). By random mutagenesis, Zenno et al generated a number of nfsb mutants that had greatly increased flavin reductase activity. These mutants all had substitutions of phenylalanine 124 (F124), a crucial position in the αG helix. F124 mutants having substitutions with serine, alanine, threonine, leucine, valine, isoleucine, aspartate, glutamine, arginine and histidine were generated, all of which had substantially increased flavin reductase activity. However, with one exception, the nitroreductase activity of these mutants was either broadly similar or substantially reduced, as judged with nitrofurazone and nitrofurantoin as substrates. The histidine mutant (F124H) had approximately double the wild-type activity for these substrates. However, firstly, these disclosures give no information as to what the effects on other substrates, such as CB1954, might be. Secondly, such data as are disclosed suggest that mutations of the F124 position have, at best, an unpredictable effect on nitroreductase activity and, in general, a deleterious effect.

International patent application WO 00/47725 (Minton et al) discloses bacterial nitroreductases that are structurally unrelated to the E.coli NFSB-encoded enzyme and that are derived from Bacillus species.

The aim of GDEPT is to obtain efficient conversion of a prodrug such as CB1954 in target cells in order to kill not only NTR-expressing cells but also bystander tumour cells that may not have been successfully transfected or transduced. It is therefore desirable to have efficient delivery of the NTR-encoding DNA, prodrugs with as high a therapeutic index as possible, and a nitroreductase enzyme that is as efficient as possible in the conversion of CB1954 and other nitro-based prodrugs to toxic DNA cross-linking products. To address the latter, it is desirable to develop modified nitroreductase enzymes, since these would allow more efficient therapy and/or lower systemic doses of the prodrug. Although prodrugs are of relatively low toxicity in comparison with their activated derivatives, it is nevertheless desirable to reduce the chances of adverse effects by minimising the required dose.

### STATEMENT OF INVENTION

Throughout the description and claims of this specification, the words “comprise” and “contain” and variations of the words, for example “comprising” and “comprises”, means “including but not limited to”, and is not intended to (and does not) exclude other moieties, substitutions, modifications, additives, components, integers or steps.

It is to be understood that references to ‘cancer’ and treatment of cancer, equally apply to a range of neoplastic, hyperplastic or other proliferative disorders including, but not limited to: carcinomas, sarcomas, melanomas, lymphomas, leukaemias and other lymphoproliferative or myeloproliferative conditions, and benign hyperplasias, (such as benign prostatic enlargement).

The present invention is based on efforts to produce a nitroreductase with improved activity in the reduction of prodrugs, especially CB1954. The invention provides mutants of the E. coli nitroreductase enzyme (EC1.6.99.7, alternatively known as the oxygen insensitive NAD(P)H nitroreductase or dihydropteridine reductase) encoded by the NFSB gene (alternatively known as NFNB, NFSI, or DPRA) that have significantly greater nitroreductase activity than the wild-type enzyme when assayed with CB1954.

Among these are enzymes with point mutations at position 40 (S40), in particular, serine substitution to alanine (S40A), glycine (S40G) and threonine (S40T); position 41 (T41), in particular, threonine substitutions to asparagine (T41N), glycine (T41G), isoleucine (T41 1), leucine (T41L) and serine (T41S); position 68 (Y68), in particular, tyrosine substitutions to alanine (Y68A), asparagine (Y68N), aspartate (Y68D), cysteine (Y68C), glutamine (Y68Q), glycine (Y68G), histidine (Y68H), serine (Y68S), and tryptophan (Y68W); position 70 (F70), in particular, phenylalanine substitutions to alanine (F70A), cysteine (F70C), glutamine (F70Q), glutamate (F70E), glycine (F70G), isoleucine (F70l), leucine (F70L), proline (F70P), serine (F70S), threonine (F70T) and valine ((F70V); position 71(N71), in particular, asparagine substitutions to aspartate (N71D), glutamine (N71Q) and serine (N71S); position 120 (G120), in particular, glycine substitutions to alanine (G120A), serine (G120S) and threonine (G120T). Of particular interest is a group of mutations centred on position 124. Phenylalanine substitutions to alanine (F124A), asparagine (F124N), cysteine (F124C), glutamine (F124Q), glycine (F124G), histidine (F124H), isoleucine (F124l), leucine (F124L), lysine (F124K), methionine (F124M), serine (F124S), threonine (F124T), tryptophan (F124W), tyrosine (F124Y) and valine (F124V) are all shown to result in mutant enzymes with substantially greater activity with CB 1954 than the wild-type.

In addition to disclosing single mutants, a number of multiply-mutated recombinant NTRs are provided. Double mutants of tyrosine 68 (Y68) and phenylalanine 124 (F124) were found to have greater activity, especially a tyrosine 68 to glycine substitution combined with a phenylalanine 124 to tryptophan substitution (giving mutant Y68G/F124W). Also beneficial is the double mutant comprising an asparagine 71 to serine substitution combined with a phenylalanine 124 to lysine substitution (giving mutant N71S/F124K). Such improved enzymes are especially useful in directed enzyme prodrug therapy. In particular, a polynucleotide comprising a sequence encoding the improved nitroreductase, together with a promoter and such other regulatory elements required to express said encoded nitroreductase, may be included in a vector suitable for gene therapy. Such a vector may be a plasmid vector, whether intended to replicate episomally, to be transiently expressed, or to integrate into the target cell genome.

Among the regulatory elements operably linked to the encoded enzyme may be elements facilitating tissue-specific expression, such as locus control regions (see U.S. Pat. No. 5,736,359, which is incorporated herein by reference, or EP 0 332667) elements facilitating activation of transcription in most or all tissues, such as ubiquitous chromatin opening elements (see WO 00/05393, U.S. application Ser. No. 09/358,082, incorporated herein by reference ). The use of a tissue-specific promoter, enhancer or LCR, or combination thereof, may allow targeted expression of an operably-linked gene, such as one encoding a prodrug-converting enzyme, in cells of a particular tissue type. In some cases, tumour cells may be targeted in a similar way, using promoters that allow expression only in, for example, foetal tissue and certain tumour types. Use of such systems helps to prevent expression of therapeutic genes, such as prodrug-converting enzymes, in healthy tissue and so minimises adverse side-effects.

Alternatively, the vector may be a viral vector, such as adenovirus, adeno-associated virus, herpesvirus, vaccinia, or a retrovirus, including those of the lentivirus group. Such a virus may be modified to alter its natural tropism or to target it to a particular organ, tissue or cell type. In some forms of VDEPT, the specificity of the cell targeting is derived from such manipulation. Alternatively, a targeting moiety such as an antibody, or portion thereof (in which case the procedure is sometimes known as antibody-directed enzyme-prodrug therapy, or ADEPT), or some other specific ligand capable of binding to a cell surface receptor may be used to target either an active enzyme or a polynucleotide encoding such an enzyme to a target cell.

The vector may be administered to the patient systemically (parenterally or enterally), regionally (for instance by perfusion of an isolated limb, or peritoneal infusion), or locally as, for example, a direct intradermal, intramuscular, intraperitoneal, intracranial or intratumoral injection.

After administration of the polynucleotide encoding the improved nitroreductase enzyme, and allowance of a suitable time for expression of the enzyme to occur, a suitable prodrug is administered, either locally (for instance around a tumour), regionally (for instance by perfusion of an isolated limb, or peritoneal infusion) or systemically. In principle, any prodrug that is capable of being activated by means of reduction and, in particular reduction of nitro-groups, may be suitable. Such compounds include nitrobenzamides, in particular nitro- and dinitrobenzamide aziridines and mustards. Particularly suitable are the dinitrobenzamide aziridine 5-(aziridin-1-yl)-2,4-dinitrobenzamide (CB1954) and the dinitrobenzamide mustard 5-[N,N-bis (2-chloroethyl)amino]-2,4-dinitrobenzamide (SN23862), and functional and structural analogues thereof.

Accordingly, the current invention provides a recombinant mutant nitroreductase, characterised in that said nitroreductase has increased nitroreductase activity as compared to the wild-type enzyme. Preferably, said nitroreductase has an increased nitroreductase activity for prodrugs, more preferably for nitrobenzamide and dinitrobenzamide aziridine and mustard prodrugs and most preferably for the dinitrobenzamide aziridine prodrug CB1954.

In one aspect of the invention, the recombinant mutant nitroreductase is encoded by a mutated equivalent of the wild-type E. coli NFSB gene. Alternatively, the recombinant mutant nitroreductase is encoded by structurally homologous gene from another genus such as from Salmonella or Enterobacter, or from another species, such as the Salmonella typhimurium NFNB gene, or the Enterobacter cloacae NFNB gene.

In all cases is it is understood that the beneficial mutation disclosed is not exclusive of further mutations at adjacent or more distant sites in the amino acid sequence.

Accordingly is provided a recombinant mutant nitroreductase encoded by a mutated equivalent of the E.coli NFSB gene, characterised in that it comprises a substitution of one or more amino acids selected from a group consisting of serine 40, threonine 41, tyrosine 68, phenylalanine 70, asparagine 71, glycine 120, and phenylalanine 124.

A first preferred embodiment is a nitroreductase encoded by a mutated equivalent of the E.coli NFSB gene, characterised in that it comprises a substitution of serine 40 with an amino acid selected from a group consisting of alanine, glycine and threonine.

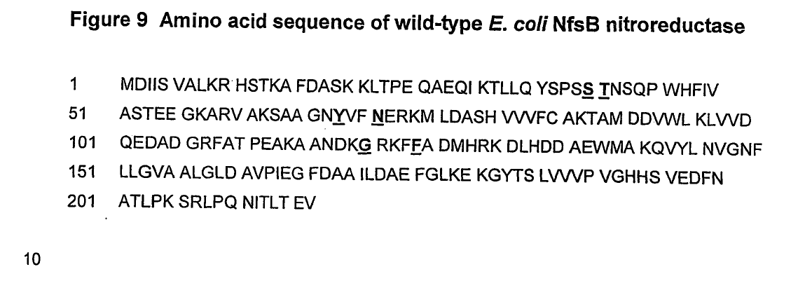

Alternatively, the nitroreductase is a protein selected from the group consisting of:
i. a recombinant E coli NFSB nitroreductase mutant corresponding to the wild type sequence of FIG. 9 (SEQ ID NO:1), characterised in that serine 40 is substituted by an amino acid selected from the group consisting of alanine, glycine and threonine;
ii. variants of (i) characterised in that they have substitutions, insertions or deletions at residues other than serine 40 and having nitroreductase activity greater than that of the wild-type protein.

A second preferred embodiment is a nitroreductase encoded by a mutated equivalent of the E.coli NFSB gene, characterised in that it comprises a substitution of threonine 41 with an amino acid selected from a group consisting of asparagine, glycine, isoleucine, leucine and serine.

Alternatively, the nitroreductase is a protein selected from the group consisting of:
i. a recombinant E coli NFSB nitroreductase mutant corresponding to the wild type sequence of FIG. 9 (SEQ ID NO:1), characterised in that threonine 41 is substituted by an amino acid selected from the group consisting of asparagine, glycine, isoleucine, leucine and serine;
ii. variants of (i) characterised in that they have substitutions, insertions or deletions at residues other than threonine 41 and having nitroreductase activity greater than that of the wild-type protein.

A third preferred embodiment is a nitroreductase encoded by a mutated equivalent of the E.coli NFSB gene, characterised in that it comprises a substitution of tyrosine 68 with an amino acid selected from a group consisting of alanine, asparagine, aspartate, cysteine, glutamine, glycine, histidine, serine, and tryptophan.

Alternatively, the nitroreductase is a protein selected from the group consisting of:
i. a recombinant E coli NFSB nitroreductase mutant corresponding to the wild type sequence of FIG. 9 (SEQ ID NO:1), characterised in that tyrosine 68 is substituted by an amino acid selected from the group consisting of alanine, asparagine, aspartate, cysteine, glutamine, glycine, histidine, serine, and tryptophan;
ii. variants of (i) characterised in that they have substitutions, insertions or deletions at residues other than tyrosine 68 and having nitroreductase activity greater than that of the wild-type protein.

Preferably, said tyrosine 68 mutant variants described in (ii) above are double mutants also comprising mutations at phenylalanine 124. More preferably, said tyrosine 68 and phenylalanine 124 double mutants comprise a first substitution of tyrosine 68 to glycine (Y68G) and a second substitution of phenylalanine 124 by an amino acid selected from either one of glutamine (F124Q) or tryptophan (F124W).

A fourth preferred embodiment is a nitroreductase encoded by a mutated equivalent of the E.coli NFSB gene, characterised in that it comprises a substitution of phenylalanine 70 with an amino acid selected from a group consisting of alanine, cysteine, glutamine, glutamate, glycine, isoleucine, leucine, proline, serine, threonine and valine.

Alternatively, the nitroreductase is a protein selected from the group consisting of:
i. a recombinant E coli NFSB nitroreductase mutant corresponding to the wild type sequence of FIG. 9 (SEQ ID NO:1), characterised in that phenylalanine 70 is substituted by an amino acid selected from the group consisting of alanine, cysteine, glutamine, glutamate, glycine, isoleucine, leucine, proline, serine, threonine and valine;
ii. variants of (i) characterised in that they have substitutions, insertions or deletions at residues other than phenylalanine 70 and having nitroreductase activity greater than that of the wild-type protein.

A fifth preferred embodiment is a nitroreductase encoded by a mutated equivalent of the E.coli NFSB gene, characterised in that it comprises a substitution of asparagine 71 with an amino acid selected from a group consisting of aspartate, glutamine and serine.

Alternatively, the nitroreductase is a protein selected from the group consisting of:
i. a recombinant E coli NFSB nitroreductase mutant corresponding to the wild type sequence of FIG. 9 (SEQ ID NO:1), characterised in that asparagine 71 is substituted by an amino acid selected from the group consisting of aspartate, glutamine and serine;
ii. variants of (i) characterised in that they have substitutions, insertions or deletions at residues other than asparagine 71 and having nitroreductase activity greater than that of the wild-type protein.

Preferably, said asparagine 71 mutant variants described in (ii) above are double mutants also comprising mutations at phenylalanine 124. More preferably, said asparagine 71 and phenylalanine 124 double mutants comprise a first substitution of asparagine 71 to serine (N71S) and a second substitution of phenylalanine 124 to lysine (F124K).

A sixth preferred embodiment is a nitroreductase encoded by a mutated equivalent of the E.coli NFSB gene, characterised in that it comprises a substitution of glycine 120 with an amino acid selected from a group consisting of alanine, serine and threonine.

Alternatively, the nitroreductase is a protein selected from the group consisting of:
i. a recombinant E coli NFSB nitroreductase mutant corresponding to the wild type sequence of FIG. 9 (SEQ ID NO:1), characterised in that glycine 120 is substituted by an amino acid selected from the group consisting of alanine, serine and threonine;
ii. variants of (i) characterised in that they have substitutions, insertions or deletions at residues other than glycine 120 and having nitroreductase activity greater than that of the wild-type protein.

A seventh preferred embodiment is a nitroreductase encoded by a mutated equivalent of the E.coli NfsB gene, characterised in that it comprises a substitution of phenylalanine 124 with an amino acid selected from a group consisting of asparagine, cysteine, glycine, lysine, methionine, tryptophan and tyrosine.

Alternatively, the nitroreductase is a protein selected from the group consisting of:
i. a recombinant E coli NFSB nitroreductase mutant corresponding to the wild type sequence of FIG. 9 (SEQ ID NO:1), characterised in that phenylalanine 124 is substituted by an amino acid selected from the group consisting of asparagine, cysteine, glycine, lysine, methionine, tryptophan and tyrosine;
ii. variants of (i) characterised in that they have substitutions, insertions or deletions at residues other than phenylalanine 124 and having nitroreductase activity greater than that of the wild-type protein.

In another aspect of the invention, a polynucleotide encoding any of the above mutated nitroreductases is provided.

The invention also provides a recombinant mutated nitroreductase as disclosed above, or a polynucleotide encoding it, for use as a medicament. Preferably, that medicament is of use in the treatment of cancer, more preferably by the conversion of a prodrug to an active cytotoxic compound, and further preferably the prodrug to be converted to an active cytotoxic compound is a nitrobenzamide aziridine or mustard, and most preferably it is CB1954.

A eighth preferred embodiment of the invention is a recombinant mutant nitroreductase encoded by a mutated E.coli NfsB gene, characterised in that it comprises the substitution of phenylalanine 124 with an amino acid selected from the group consisting of alanine, glutamine, histidine, isoleucine, leucine, serine, threonine or valine, for use as a medicament. Preferably, that medicament is of use in the treatment of cancer, or other proliferative disorder, more preferably by the conversion of a prodrug to an active cytotoxic compound, and further preferably the prodrug to be converted to an active cytotoxic compound is a nitrobenzamide aziridine or mustard, and most preferably it is CB1954.

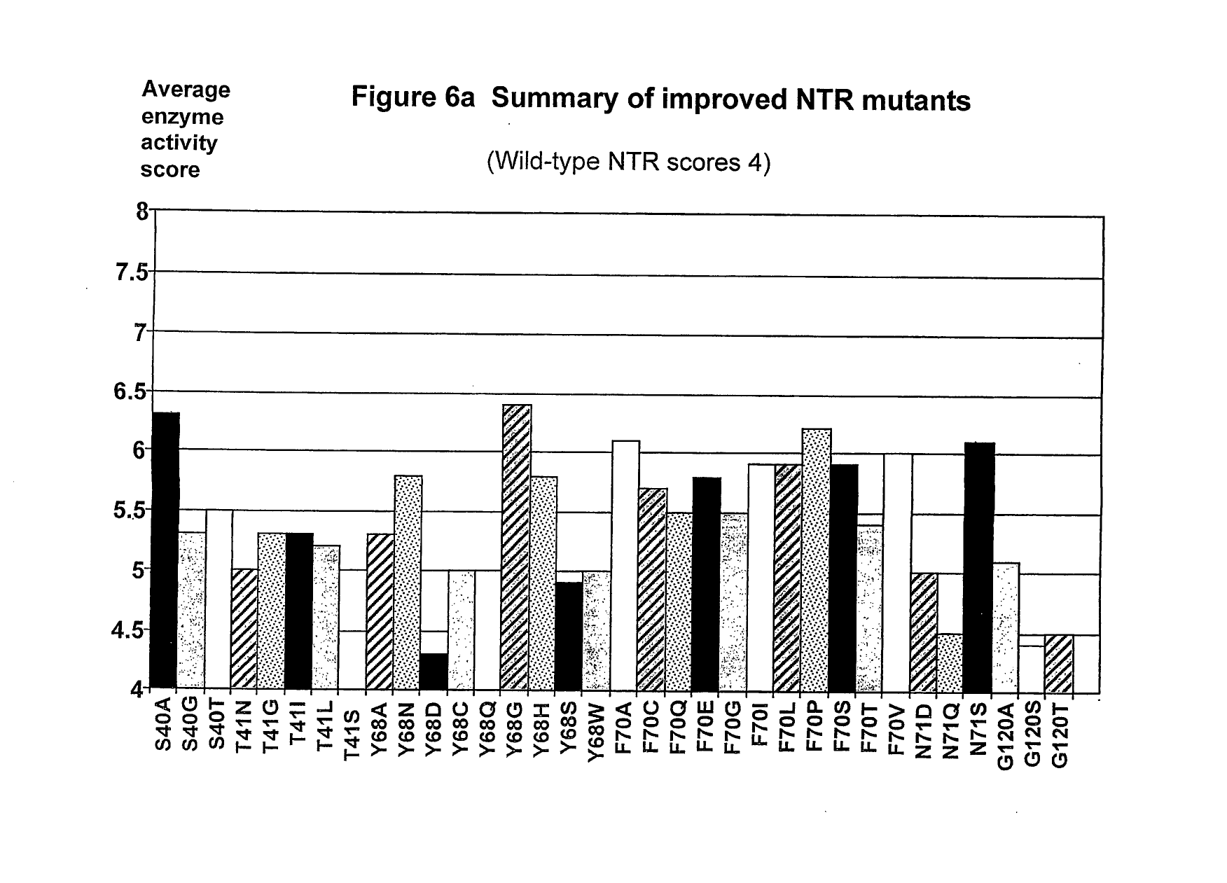

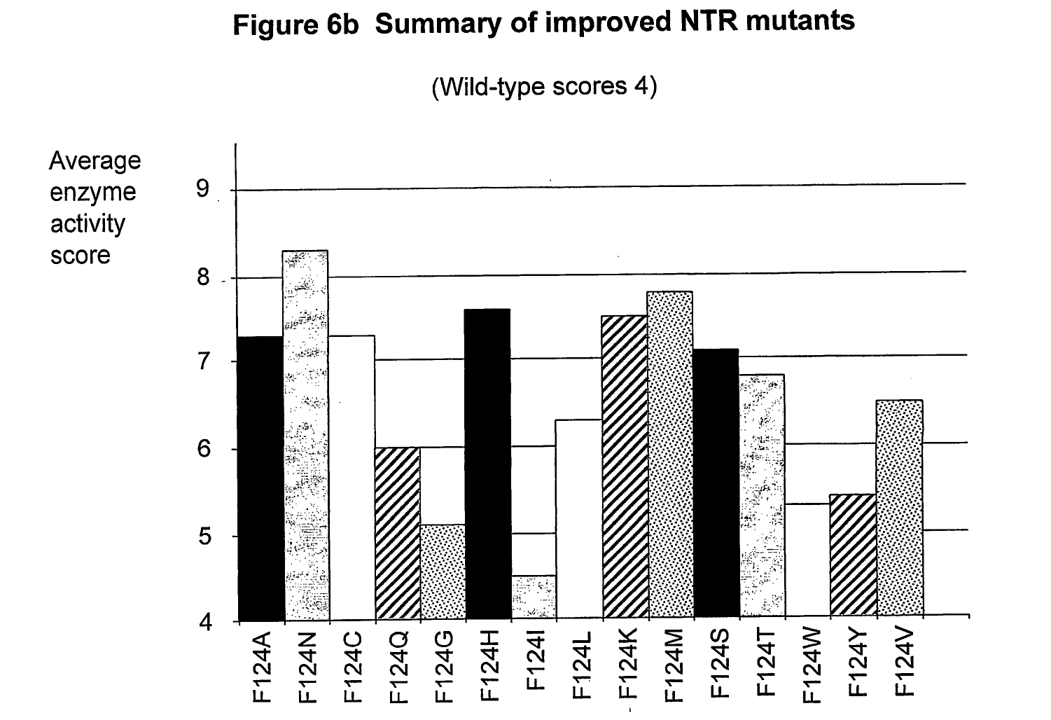

Alternatively the nitroreductase is a protein selected from the group consisting of:
i. A recombinant E coli NfsB nitroreductase mutant corresponding to the wild type sequence of FIG. 6, characterised in that phenylalanine 124 is substituted by an amino acid selected from the group consisting of alanine, glutamine, histidine, isoleucine, leucine, serine, threonine or valine;
ii. Variants of (i) characterised in that they have substitutions, insertions or deletions at residues other than phenylalanine 124 and having nitroreductase activity greater than that of the wild-type protein.
   for use as a medicament. Preferably, that medicament is of use in the treatment of cancer, more preferably by the conversion of a prodrug to an active cytotoxic compound, and further preferably the prodrug to be converted to an active cytotoxic compound is a nitrobenzamide aziridine or mustard, and most preferably it is CB1954.

In another aspect, the use of any of the above-disclosed recombinant mutant nitroreductases and polynucleotides encoding them for the manufacture of a medicament is disclosed. Preferably, said medicament is for enzyme prodrug therapy. Said medicament may take the form of naked DNA, a DNA-peptide, DNA-lipid or DNA-polymer conjugate or complex, or viral vector, comprising a polynucleotide encoding a recombinant mutant nitroreductase operably linked to a promoter with or without further elements such as enhancers and LCRs so arranged as to permit efficient tissue-specific expression of said nitroreductase in the appropriate cells following administration and transfection of said cells. Alternatively, said medicament may comprise such a DNA-peptide, DNA-lipid or DNA-polymer conjugate or complex, or viral vector comprising a targeting moiety, such as an antibody or fragment thereof, or a peptide or carbohydrate ligand capable of binding specifically to a suitable cell surface receptor or other structure so as to allow efficient targeting to appropriate cell types.

Also provided is a process to manufacture a medicament characterised in the use of any of the above-disclosed recombinant mutant nitroreductases and polynucleotides encoding them.

In another embodiment is provided a pharmaceutical composition comprising any one of the above-disclosed recombinant mutant nitroreductases or polynucleotides encoding them, or viral or non-viral vectors comprising such polynucleotides in an acceptable diluent or excipient.

In another aspect of the invention are provided vectors comprising isolated polynucleotides encoding one or more of the above-disclosed recombinant mutant nitroreductases. As detailed below, these vectors may be replicating or non-replicating, episomal or integrating, designed for use in prokaryotic or eukaryotic cells. They may be expression vectors providing ubiquitous or tissue-specific expression of the encoded nitroreductase, which may be operably-linked to suitable promoters and other elements required for appropriate expression, such as LCRs or UCOEs. In a more preferred embodiment, said vector provides tissue-specific expression of nitroreductase. Further preferably, the nitroreductase is preferentially expressed in tumours. Most preferably, the vector comprises a TCF-responsive element operably linked to a polynucleotide encoding nitroreductase.

In a further preferred embodiment, said vector is a virus, and most preferably it is an adenovirus. The use of adenovirus vectors comprising a TCF-responsive tumour-selective promoter element operably linked to a nitroreductase gene is described in International application number PCT/GB01/00856, the whole of which is incorporated herein by reference. A copy of GB 01/00856 is filed with this application and its content is included in the present application but the copy is not included in the published specification of this application.

The vector may be any vector capable of transferring DNA to a cell. Preferably, the vector is an integrating vector or an episomal vector.

Preferred integrating vectors include recombinant retroviral vectors. A recombinant retroviral vector will include DNA of at least a portion of a retroviral genome which portion is capable of infecting the target cells. The term “infection” is used to mean the process by which a virus transfers genetic material to its host or target cell. Preferably, the retrovirus used in the construction of a vector of the invention is also rendered replication-defective to remove the effect of viral replication of the target cells. In such cases, the replication-defective viral genome can be packaged by a helper virus in accordance with conventional techniques. Generally, any retrovirus meeting the above criteria of infectiousness and capability of functional gene transfer can be employed in the practice of the invention.

Suitable retroviral vectors include but are not limited to pLJ, pZip, pWe and pEM, well known to those of skill in the art. Suitable packaging virus lines for replication-defective retroviruses include, for example, ΨCrip, ΨCre, Ψ2 and ΨAm.

Other vectors useful in the present invention include adenovirus, adeno-associated virus, SV40 virus, vaccinia virus, HSV and poxvirus vectors. A preferred vector is the adenovirus. Adenovirus vectors are well known to those skilled in the art and have been used to deliver genes to numerous cell types, including airway epithelium, skeletal muscle, liver, brain and skin (Hitt, M M, Addison C L and Graham, F L (1997) Human adenovirus vectors for gene transfer into mammalian cells. Advances in Pharmacology, 40: 137-206; and Anderson W F (1998) Human gene therapy. Nature, 392: (6679 Suppl): 25-30).

A further preferred vector is the adeno-associated (AAV) vector. AAV vectors are well known to those skilled in the art and have been used to stably transduce human T-lymphocytes, fibroblasts, nasal polyp, skeletal muscle, brain, erythroid and haematopoietic stem cells for gene therapy applications (Philip et al., 1994, Mol. Cell. Biol., 14, 2411-2418; Russell et al., 1994, PNAS USA, 91, 8915-8919; Flotte et al., 1993, PNAS USA, 90, 10613-10617; Walsh et al., 1994, PNAS USA, 89, 7257-7261; Miller et al., 1994, PNAS USA, 91, 10183-10187; Emerson, 1996, Blood, 87, 3082-3088). International Patent Application WO 91/18088 describes specific AAV based vectors.

Preferred episomal vectors include transient non-replicating episomal vectors and self-replicating episomal vectors with functions derived from viral origins of replication such as those from EBV, human papovavirus (BK) and BPV-1. Such integrating and episomal vectors are well known to those skilled in the art and are fully described in the body of literature well known to those skilled in the art. In particular, suitable episomal vectors are described in WO98/07876.

Mammalian artificial chromosomes can also be used as vectors in the present invention. The use of mammalian artificial chromosomes is discussed by Calos (1996, TIG, 12, 463-466).

In a preferred embodiment, the vector of the present invention is a plasmid. The plasmid may be a non-replicating, non-integrating plasmid.

The term “plasmid” as used herein refers to any nucleic acid encoding an expressible gene and includes linear or circular nucleic acids and double or single stranded nucleic acids. The nucleic acid can be DNA or RNA and may comprise modified nucleotides or ribonucleotides, and may be chemically modified by such means as methylation or the inclusion of protecting groups or cap- or tail structures.

A non-replicating, non-integrating plasmid is a nucleic acid which when transfected into a host cell does not replicate and does not specifically integrate into the host cell's genome (i.e. does not integrate at high frequencies and does not integrate at specific sites).

Replicating plasmids can be identified using standard assays including the standard replication assay of Ustav et al., EMBO J., 10, 449-457, 1991.

The present invention also provides a host cell transfected with the vector of the present invention. The host cell may be any mammalian cell. Preferably the host cell is a rodent or mammalian cell. Most preferably it is a human cell.

Numerous techniques are known and are useful according to the invention for delivering the vectors described herein to cells, including the use of nucleic acid condensing agents, electroporation, complexing with asbestos, polybrene, DEAE cellulose, Dextran, liposomes, cationic liposomes, lipopolyamines, polyornithine, particle bombardment and direct microinjection (reviewed by Kucherlapati and Skoultchi, Crit. Rev. Biochem. 16:349-379 (1984); Keown et al., Methods Enzymol. 185:527 (1990)).

A vector of the invention may be delivered to a host cell non-specifically or specifically (i.e., to a designated subset of host cells) via a viral or non-viral means of delivery. Preferred delivery methods of viral origin include viral particle-producing packaging cell lines as transfection recipients for the vector of the present invention into which viral packaging signals have been engineered, such as those of adenovirus, herpes viruses and papovaviruses. Preferred non-viral based gene delivery means and methods may also be used in the invention and include direct naked nucleic acid injection, nucleic acid condensing peptides and non-peptides, cationic liposomes and encapsulation in liposomes.

The direct delivery of vector into tissue has been described and some short-term gene expression has been achieved. Direct delivery of vector into muscle (Wolff et al., Science, 247, 1465-1468, 1990) thyroid (Sykes et al., Human Gene Ther., 5, 837-844, 1994) melanoma (Vile et al., Cancer Res., 53, 962-967, 1993), skin (Hengge et al., Nature Genet, 10, 161-166, 1995), liver (Hickman et al., Human Gene Therapy, 5, 1477-1483, 1994) and after exposure of airway epithelium (Meyer et al., Gene Therapy, 2, 450-460, 1995) is clearly described in the prior art.

Various peptides derived from the amino acid sequences of viral envelope proteins have been used in gene transfer when co-administered with polylysine DNA complexes (Plank et al., J. Biol. Chem. 269:12918-12924 (1994));. Trubetskoy et al., Bioconjugate Chem. 3:323-327 (1992); WO 91/17773; WO 92/19287; and Mack et al., Am. J. Med. Sci. 307:138-143 (1994)) suggest that co-condensation of polylysine conjugates with cationic lipids can lead to improvement in gene transfer efficiency. International Patent Application WO 95/02698 discloses the use of viral components to attempt to increase the efficiency of cationic lipid gene transfer.

Nucleic acid condensing agents useful in the invention include spermine, spermine derivatives, histones, cationic peptides, cationic non-peptides such as polyethyleneimine (PEI) and polylysine. ‘Spermine derivatives’ refers to analogues and derivatives of spermine and include compounds as set forth in International Patent Application WO 93/18759 (published Sep. 30, 1993).

Disulphide bonds have been used to link the peptidic components of a delivery vehicle (Cotten et al., Meth. Enzymol. 217:618-644 (1992)); see also, Trubetskoy et al. (supra).

Delivery vehicles for delivery of DNA constructs to cells are known in the art and include DNA/poly-cation complexes which are specific for a cell surface receptor, as described in, for example, Wu and Wu, J. Biol. Chem. 263:14621 (1988); Wilson et al., J. Biol. Chem. 267:963-967 (1992); and U.S. Pat. No. 5,166,320).

Delivery of a vector according to the invention is contemplated using nucleic acid condensing peptides. Nucleic acid condensing peptides, which are particularly useful for condensing the vector and delivering the vector to a cell, are described in International Patent Application WO 96/41606. Functional groups may be bound to peptides useful for delivery of a vector according to the invention, as described in WO 96/41606. These functional groups may include a ligand that targets a specific cell-type such as a monoclonal antibody, insulin, transferrin, asialoglycoprotein, or a sugar. The ligand thus may target cells in a non-specific manner or in a specific manner that is restricted with respect to cell type.

The functional groups also may comprise a lipid, such as palmitoyl, oleyl, or stearoyl; a neutral hydrophilic polymer such as polyethylene glycol (PEG), or polyvinylpyrrolidine (PVP); a fusogenic peptide such as the HA peptide of influenza virus; or a recombinase or an integrase. The functional group also may comprise an intracellular trafficking protein such as a nuclear localisation sequence (NLS), an endosome escape signal such as a membrane disruptive peptide, or a signal directing a protein directly to the cytoplasm.

Also provided is a host cell comprising a polynucleotide encoding a recombinant mutant nitroreductase of the invention, or a host cell comprising a vector comprising such a polynucleotide. Such a host cell may be a bacterial cell used to grow, manufacture, screen and test said vector, or a eukaryotic cell, preferably a mammalian cell and most preferably a human cell, in which the encoded nitroreductase is expressed.

In another aspect of the invention is provided an isolated polynucleotide encoding a nitroreductase of the invention, or a vector comprising such a polynucleotide, or a host cell comprising either said polynucleotide or vector for use in gene therapy. Preferably such gene therapy is of use in treating cancer.

In another aspect of the invention, the use of a recombinant nitroreductase to aid in the design of, or screening for improved prodrugs is provided. Such a use comprises contacting said nitroreductase with candidate prodrugs and chemically measuring the kinetics of conversion to a reduced product. Alternatively, an in vitro assay may be used where the ability of a disclosed recombinant mutant nitroreductase to convert candidate prodrugs to cytotoxic products is assayed by the inhibition of growth of bacterial host cells in the presence of various concentrations said prodrugs, or by the killing of eukaryotic cells cultured in the presence of various concentrations of said prodrugs. This may be further examined by an in vivo assay of, for example, tumour killing in an experimental animals by administration of a polynucleotide encoding the recombinant mutant nitroreductase, allowing a suitable time for expression to occur, and then administration of various doses of candidate prodrugs. Comparison of the results using various mutants as well as wild-type nitroreductase allows identification of optimal combinations of mutant nitroreductases and novel prodrugs that provide improved efficiency and therapeutic index.

Also provided is a method of treating cancer in a mammalian subject, comprising administering any of the isolated polynucleotides or vectors described above, allowing a suitable time for expression of the encoded nitroreductase to occur, and administering a prodrug capable of being activated by said expressed nitroreductase.

### DETAILED DESCRIPTION OF THE INVENTION

The invention is described through Examples with reference to the accompanying Tables and Figures, wherein:

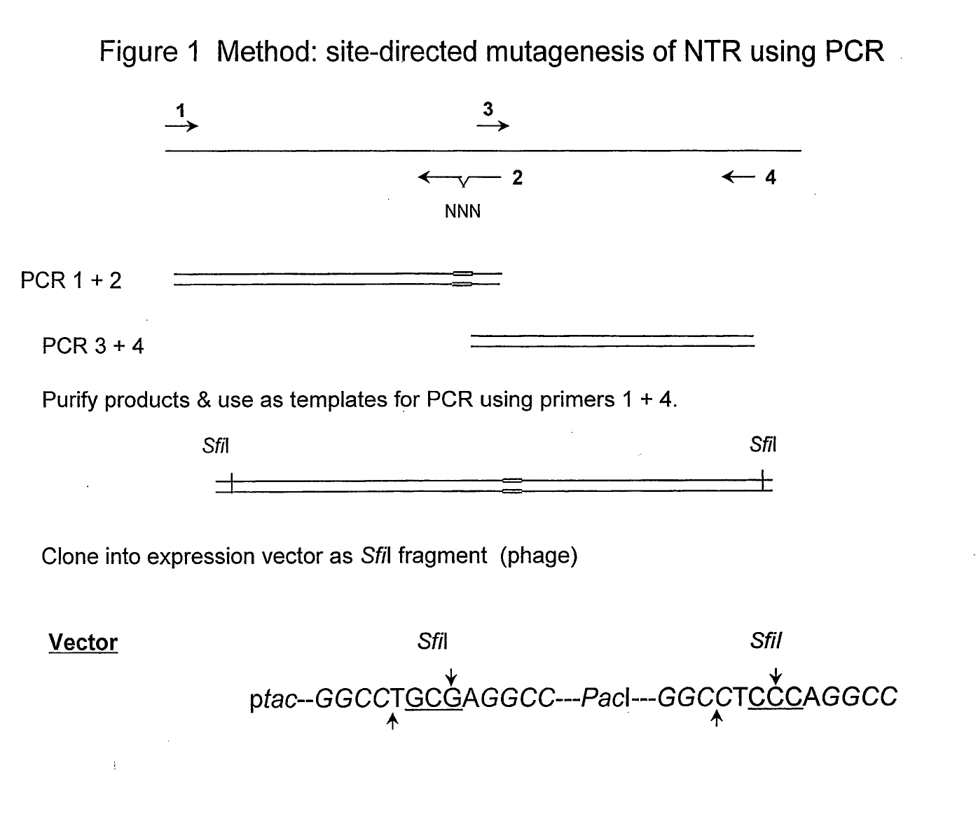

FIG. 1 illustrates the method of site-directed mutagenesis used to generate NTR mutants using PCR;

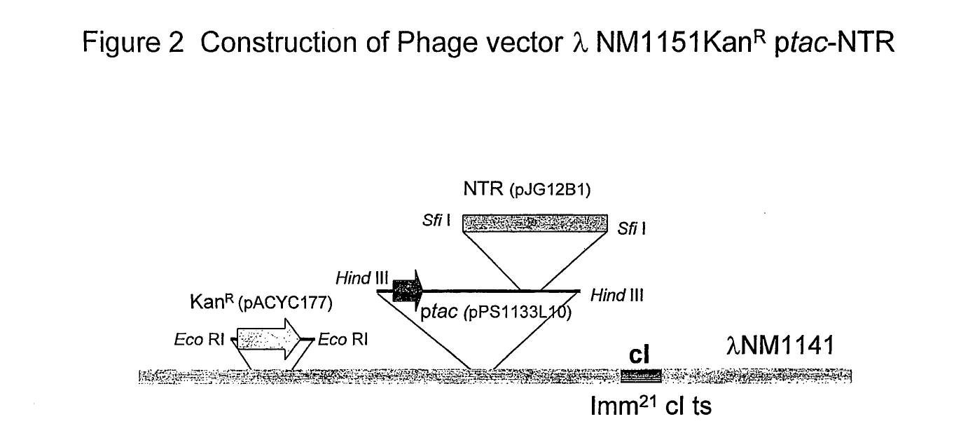

FIG. 2 shows the construction of the phage (λNM1151Kan^{R}ptac-NTR) used to express the mutant NTRs in lysogenised E. coli cells;

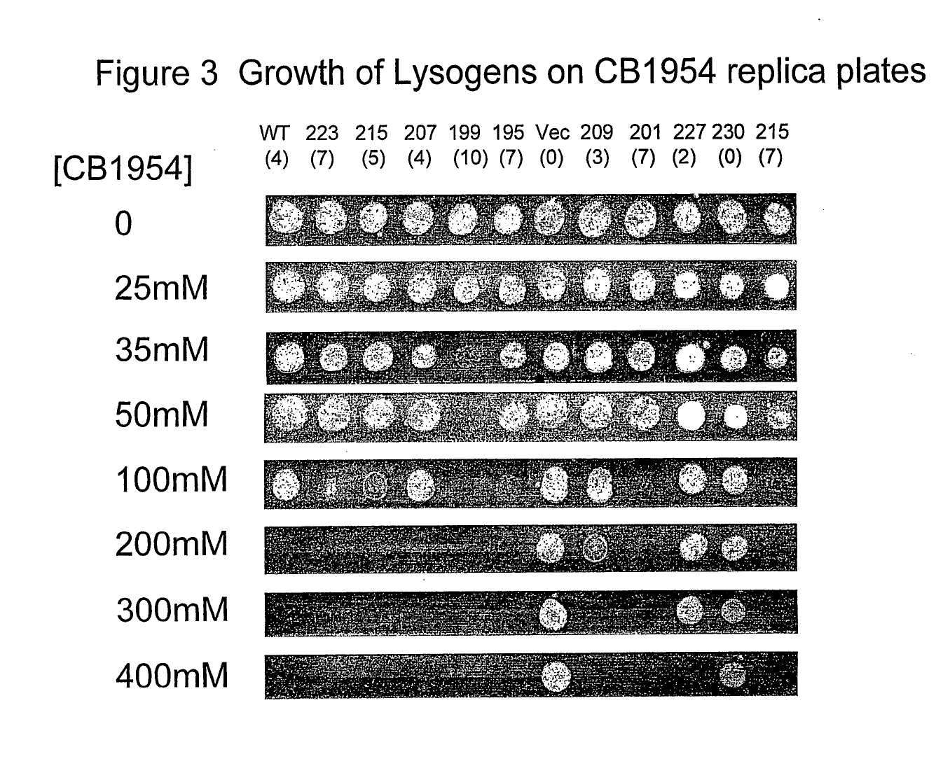

FIG. 3 shows an example of screening mutant NTR-expressing lysogens through growth on increasing concentrations of CB1954. More efficient NTRs lead to greater genotoxicity and so less growth;

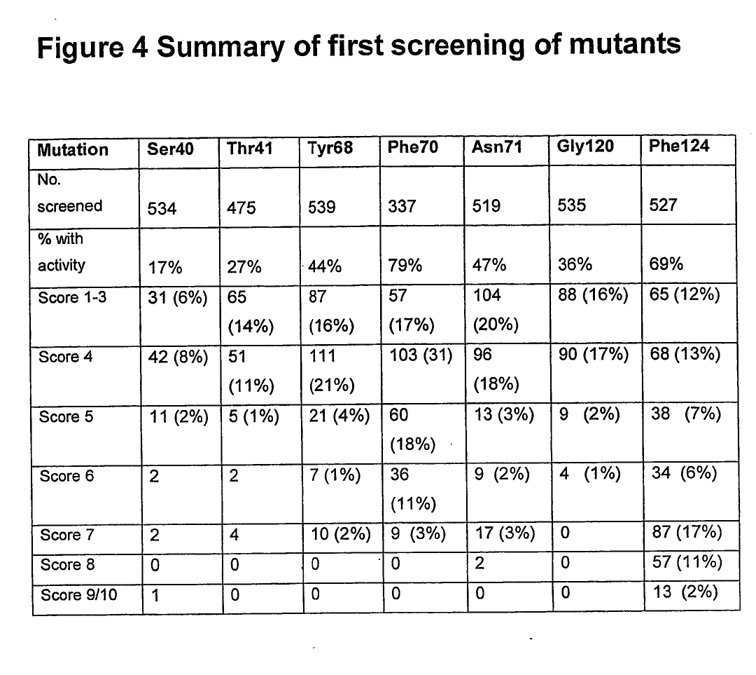

FIG. 4 shows the results of the first round of screening of mutant clones by the method illustrated in FIG. 3;

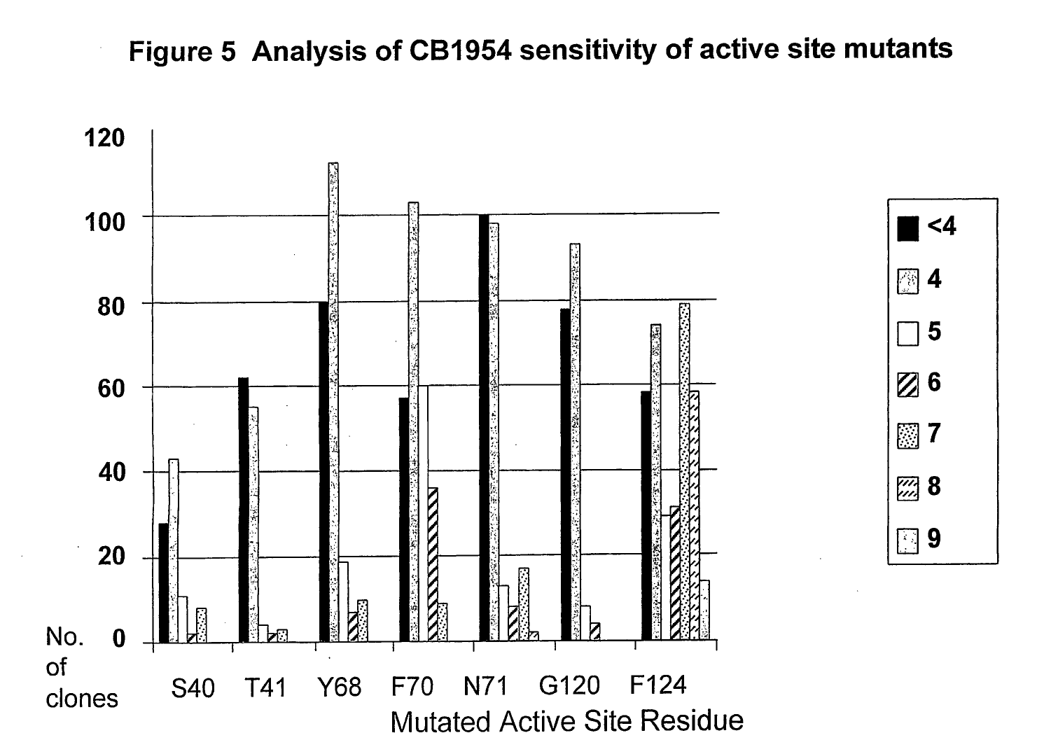

FIG. 5 shows an analysis of the number of mutants generated and whether NTR activity was increased or decreased (wild-type enzyme scores 4) by mutation of key amino acids near the active site of NTR;

FIG. 6 summarises the enzyme activity scores for mutants showing increased activity as compared with wild-type NTR, with FIG. 6a showing the results for S40, T41, Y68, F70, N71, and G120 mutants, while FIG. 6b shows the results for F124 mutants;

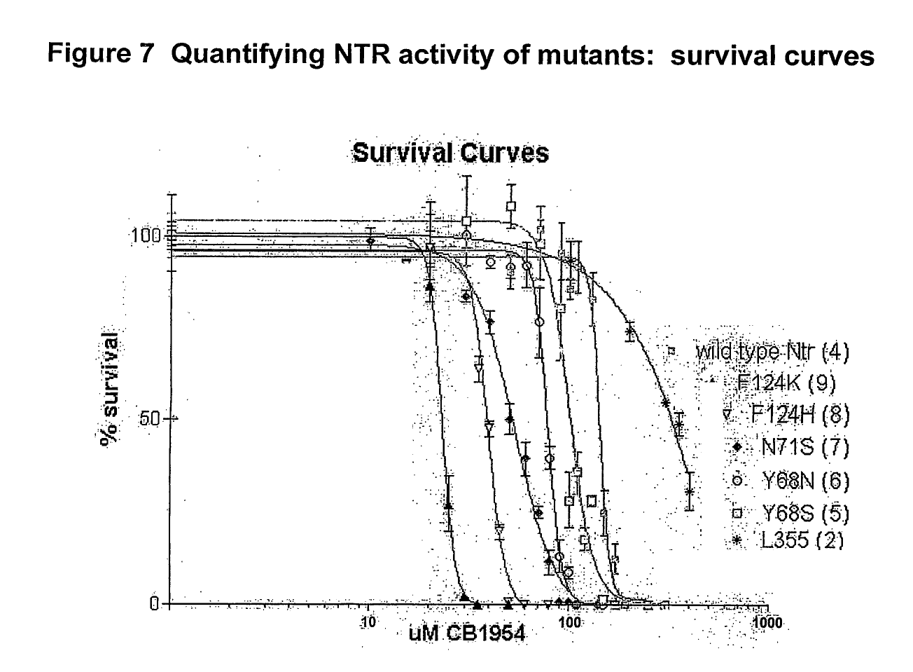

FIG. 7 shows an example of survival curves obtained for a number of mutant clones with percentage survival plotted against CB1954 concentration to enable an IC50 value to be calculated;

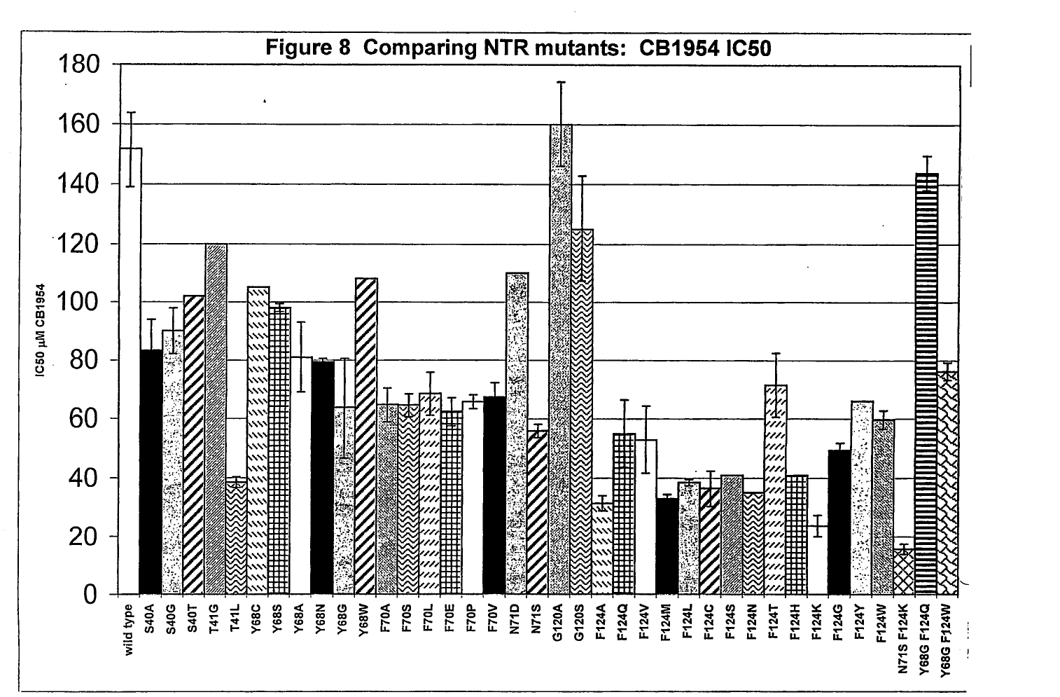

FIG. 8 represents the IC50 data generated by such experiments compared to the wild-type enzyme;

FIG. 9 shows the amino acid sequence (SEQ ID NO:1) of wild-type NTR—the protein encoded by the E coli NfsB gene. The key mutation sites at S40, T41, Y68, F70, N71, G120, and F124 are underlined and in bold.

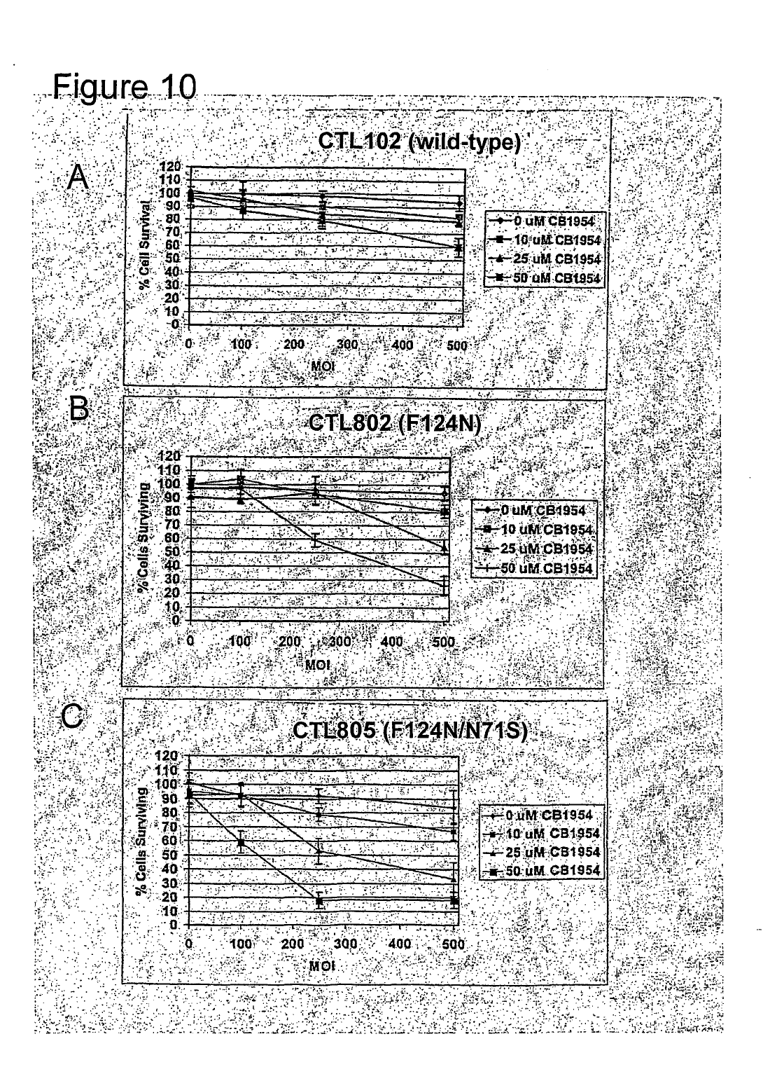

FIG. 10 shows results of experiments using three different recombinant adenovirus vectors to express wild type (A), F124N (B) or double mutant F124N/N71S (C) NTRs in mammalian cell, resulting in sensitisation to and killing by CB1954. The % cells surviving at a range of MOIs and CB1954 concentrations are shown

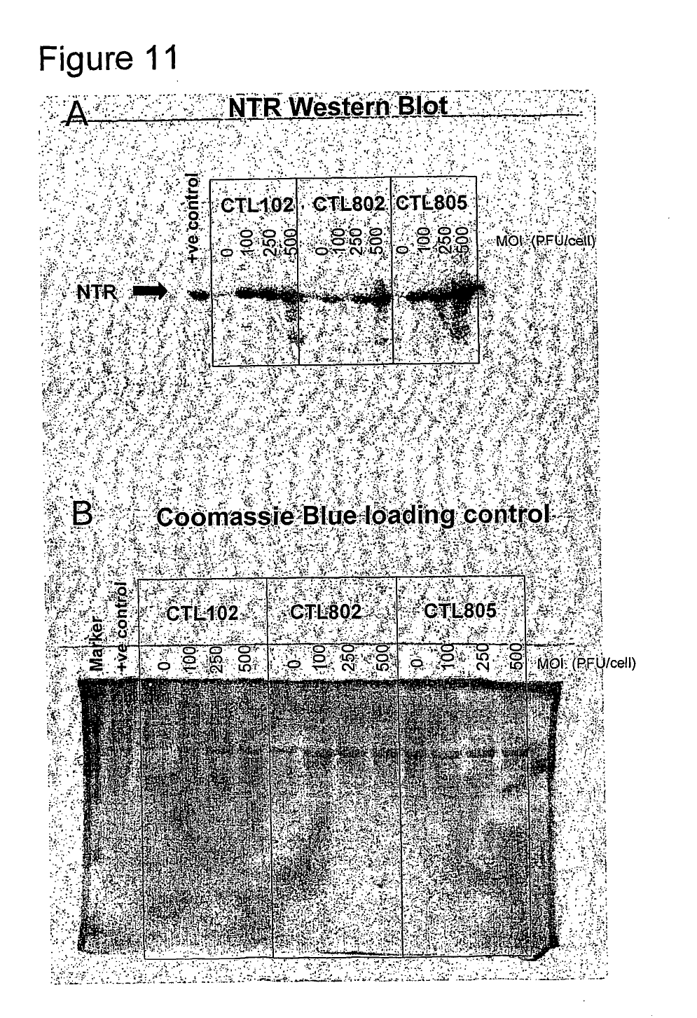

FIG. 11 shows the levels of expression of the wild type, and F124N and F124N/N71S NTR mutants by western blotting (A), with a Coomassie stained loading control (B).

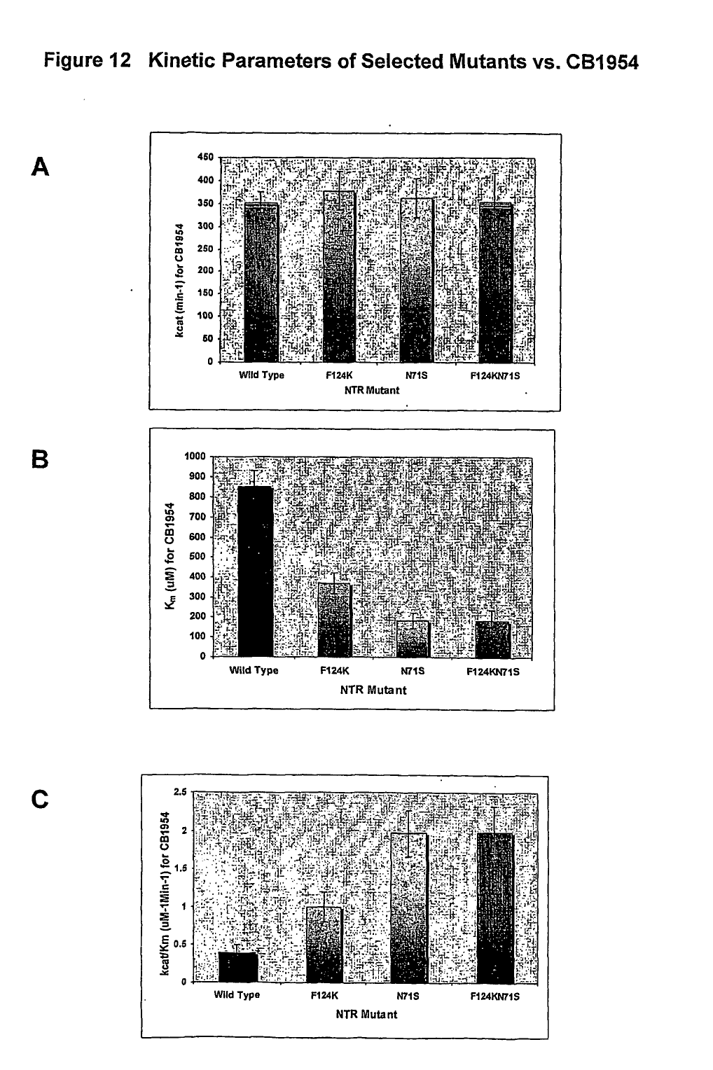

FIG. 12 shows enzyme kinetic data (k_{cat}, Kₘ, and k_{cat}/Kₘ ratio) for wild type, F124K, N71S and F124N/N71S mutants.

### EXAMPLE 1

### Generation of NTR Mutants with Increased CB 1954 Converting Activity

Methods

Mutagenesis

Mutations were introduced into the NTR sequence at various positions by PCR (see FIG. 1) using plasmid pJG12B1 as a template. This is a pUC19-derived plasmid containing the E.coli DH5α NTR within Sfi I cloning sites downstream of the tac promoter.

Referring to Table 1, for mutagenesis at position 40, primer 2 was JG126B (SEQ ID NO:6) and primer 3 was JG126A (SEQ ID NO:5); at position 41 primer 2 was JG126C (SEQ ID NO:7) and primer 3 was JG126A(SEQ ID NO:5); at position 68 primer 2 was JG127B (SEQ ID NO:9) and primer 3 was JG127A(SEQ ID NO:8); at position 71 primer 2 was JG127C (SEQ ID NO:10) and primer 3 was JG127A(SEQ ID NO:8); at position 120, primer 2 was JG128B (SEQ ID NO:12) and primer 3 was JG128A(SEQ ID NO:11); at position 124 primer 2 was JG128C (SEQ ID NO:13) and primer 3 was JG128A (SEQ ID NO:11). Primer 1 was the 5′ primer for JG14A (SEQ ID NO:2) and the 3′ primer, primer 4, was an M13 reverse sequencing primer, PS1107rev (SEQ ID NO:3) (Table 1).

After denaturation at 94° for 5 min, PCR was for 25 cycles of 94°/45s; 55°/50s; 72°/90s followed by 72°/7 min. Pfu DNA polymerase was used according to the manufacturers recommendations (Stratagene ™) to minimise additional mutations. The products of PCR using primers 1 with 2, and 3 with 4, were gel purified to remove excess primers and 5 ng of each was used as a template for PCR with primers 1 and 2 to restore a full length NTR gene.

TABLE 1

NTR Mutagenesis PCR Primers

| SEQ | | |
|---|---|---|
| ID NO | Primer | Sequence 5′ to 3′ |
| 2 | JG14A | GACAATTAATCATCGGCTCG |
| 3 | PS1107Rev | GCGGATAACAATTTCACACAGGA |
| 4 | JG2B | CAGAGCATTAGCGCAAGGTG |
| 5 | JG126A | CCCAGCCGTGGCATTTTATTGTTG |
| 6 | JG126B | CAACAATAAAATGCCACGGCTGGGAGTTGGTNN |
| | | NGGATGGGCTGTATTGC |
| 7 | JG126C | CAACAATAAAATGCCACGGCTGGGAGTTNNNGC |
| | | TGGATGGGCTGTATTGC |
| 8 | JG127A | GAGCGTAAAATGCTTGATGCCTCG |
| 9 | JG127B | CGAGGCATCAAGCATTTTACGCTCGTTGAACAC |
| | | NNNATTACCGGCAGCGG |
| 10 | JG127C | CGAGGCATCAAGCATTTTACGCTCNNNGAACAC |
| | | GTAATTACCGGC |
| 11 | JG128A | GCTGATATGCACCGTAAAGATCTGC |
| 12 | JG128B | GCAGATCTTTACGGTGCATATCAGCGAAGAACT |
| | | TGCGNNNTTTATCGTTCG |
| 14 | JG128C | GCAGATCTTTACGGTGCATATCAGCNNNGAACT |
| | | TGCG |
| 15 | JG127D | CGAGGCATCAAGCATTTTACGCTCGTTNNNCAC |
| | | GTAATTACCGGC |

λJG3J1 was produced from λNM1141 (FIG. 2) by cloning a kanamycin resistance gene from pACYC177 into an Eco RI site and the ptac promoter from pPS1133L10 (ultimately derived from pDR540 [Pharmacia] into a Hind III site. The final PCR products were digested with SfiI and the major central fragment inserted between two matching Sfi I sites within the Hind III fragment, downstream of the tac promoter.

The ligation mix was packaged (Stratagene) into lambda bacteriophage particles that were used to infect UT5600 cells (NTR ⁻). As a control wild type NTR was also cloned into this vector (JG16C2). Kanamycin resistant lysogens were selected on agar plates (30 ug/ul kanamycin) then individually grown overnight in a well of a 96-well plate in LB+Kanamycin. The clones were replica plated on to a series of plates containing Tris-buffered (50 mM, pH 7.5) LB agar with kanamycin, IPTG (0.1 mM) and CB1954 at a concentration of 0, 25, 35, 50, 100, 200, 300 or 400 μM (see FIG. 3). The plates were scored as shown in Table 2 and the results shown in FIGS. 4 and 5.

TABLE 2

| Score | Criteria |
|---|---|
| 0 | Good growth on all concentrations of CB 1954 = vector |
| 1 | Good growth on 100 and 200, faint on 300, ring of |
| | growth on 400 μM |
| 2 | Good growth on 100 and 200, ring growth on 300, |
| | none or very faint on 400 μM |
| 3 | Good growth on 100, faint to good on 200, none or |
| | very faint on 300 and 400 μM |
| 4 | Good growth on 100 μM, none to ring growth |
| | on 200 μM = wild type |
| 5 | Good growth on 50 μM, ring on 100 μM, |
| | none on 200, 300 or 400 μM |
| 6 | Faint growth on 50 μM, ring on 100 μM |
| 7 | Good growth on 50 μM, none on 100 μM |
| 8 | Ring growth on 50 μM, none on 100 μM |
| 9 | None or very faint growth on 50 μM, none on 100 μM |
| 10 | None or ring growth on 35 μM |

The DNA from clones with a score >4 was amplified by PCR using primers JG14A and JG2B (Table 1) and sequenced to determine the mutation present (ABI Prism Big Dye Terminator kit). An example of data from the first screening is shown in FIG. 4 and the results are summarised in FIG. 5. Promising clones were selected for analysis of their IC50s and the results are summarised in Table 3 below.

Combining Mutations

To generate NTR clones containing two gain-of-function mutations the PCR method shown in FIG. 1 was used as for the first round of mutagenesis. To generate a N71S F124K mutant, primer 1 was JG14A (SEQ ID NO:2) and primer 2 was PS1013A (SEQ ID NO:14) (Table 1) using 1 μl phage λ JG131H481 stock as a template. Primer 3 was JG127A (SEQ ID NO:8) and primer 4 was JG2B (SEQ ID NO:4) using λ JG131I399 as a template. The resulting products were then used as templates for primers JG14A (SEQ ID NO:2) and JG2B (SEQ ID NO:4) to generate the double-mutated NTR sequence for cloning as a SfiI fragment into λJG3J1 to give λJG139CB1. Similarly, to construct a Y658G F124Q double mutant, primers JG14A (SEQ ID NO:2) and PS1013A (SEQ ID NO:14) were used to amplify λJG131C19, and primers JG127A (SEQ ID NO:8) and JG2B (SEQ ID NO:4) were used to amplify λJG131I83 followed by PCR amplification of the products with primers JG14A (SEQ ID NO:2) and JG2B (SEQ ID NO:4) to give λJG139DC1. A Y68G F124W double mutant was constructed by amplifying λJG131C194 with primers JG14A (SEQ ID NO:2) and PS1013A (SEQ ID NO:14) and amplifying λJG131I505 with primers JG127A (SEQ ID NO:8) and JG2B (SEQ ID NO:4) followed by PCR using the products as templates for amplification with primers JG2B (SEQ ID NO:4) and JG14A (SEQ ID NO:2) to give λJG139EC12.

Survival Curve Data

To quantify the improvement in NTR activity in the clones in a less subjective way, a few clones were selected for further study by determining their survival curves. The lysogens were grown overnight in LB +kanamycin and diluted to approximately 1 cell per μl based on the OD. In duplicate, 100 μl diluted cells were plated into Tris-buffered LB plates containing kanamycin, IPTG and 0-400 μM CB1954. After 36h growth the number of colonies on each plate were counted and expressed as a percentage of the number present on the plates with no CB1954. Survival curves showing % survival versus concentration of CB 1954 were plotted (see examples in FIG. 7) and the IC50 determined as the concentration of CB 1954 which kills gives a 50% reduction in colony number (Table 3 and FIG. 8). A few clones containing mutations resulting in an enhanced sensitivity to CB 1954 were selected for further study.

Results

Enzyme Activity Assays

The first screening showed that clones showing increased sensitivity to CB 1954 over the baseline level of the wild-type had mutations clustering at a limited number of positions, notably 40, 41, 68, 70, 71, 120 and 124, as shown in FIG. 4. Of these, substitution of Phe124 was the commonest site for gain-of-function mutants. FIG. 5 summarises the average scores for the gain-of-function mutants identified. The highest activity mutants were all at position 124. FIG. 6 analyses the change in activity, both up and down, related to the site of mutation. Loss-of-function mutations were commonest at positions 68, 70, 71 ,and 120, although some a few significantly improved clones were also seen, particularly at positions 70 and 71. At position 124, gain-of-function mutants were more common.

IC50 Assays

FIG. 7 shows an example of a survival against CB 1954 concentration plot and the data are summarised in Table 3 and FIG. 8. The data are broadly consistent with the enzyme activity results, with a number of mutant scoring highly in both assays. On the basis of these results, a number of clones were selected for further study and identified as offering substantial benefits over the wild-type enzyme for applications such as GDEPT. Amongst these were T41L, Y68G, N71S, F124A, F124G, F124N, F124C, F124H, F124L, F124K, F124M, F124S, F124Q, F124T, F124V and F124W. In addition, mutations giving a more modest improvement, but at a less common site (implying perhaps a different mode of action), such as those at S40 and F70 were highlighted.

Double Mutants

Particularly striking was the activity of the double mutant N71S/F124K, with Y68G/F124W also having a significant gain of function over the wild-type. N71S/F124K shows increased enzyme activity as measured by reduced IC50 compared to either mutation alone. This shows that the mutations identified in the first round of screening can have an additive effect. However, the Y68G/F124Q mutant has decreased enzyme activity compared to either mutation alone with activity similar to that of the wild type enzyme, suggesting that combining two single gain-of-function mutations can also cancel each other out resulting in only wild-type levels of enzyme activity. A third double mutant, Y68G/F124W had an IC50 equivalent to that of the better single mutation alone thus demonstrating that combining mutations may also have a neutral effect.

TABLE 3

| Mutation | Clone | Score | IC50 μM CB1954 |
|---|---|---|---|
| Wild type | | 4 | 118 |
| S40A | K263 | 5-7 | 100 |
| S40A | K327 | 5-7 | 84 |
| S40G | K264 | 5 | 90 |
| S40T | K350 | 5 | 102 |
| T41G | L229 | 5-7 | 120 |
| T41L | L233 | 5-7 | 38 |
| Y68C | C88 | 5 | 105 |
| Y68S | C103 | 5-7 | 96 |
| Y68A | C146 | 5-7 | 81 |
| Y68N | C153 | 5-7 | 79 |
| Y68G | C194 | 7 | 43 |
| Y68W | C196 | 4-5 | 108 |
| N71D | H455 | 5 | 110 |
| N71S | H481 | 7 | 55 |
| G120A | D127 | 4-5 | 160 |
| G120S | D171 | 4-6 | 125 |
| F124Q | I83 | 8 | 39 |
| F124A | I104 | 7-9 | 20 |
| F124V | I115 | 7-9 | 37 |
| F124M | I116 | 9 | 33 |
| F124L | I136 | 7-9 | 38 |
| F124C | I138 | 7-9 | 36 |
| F124S | I211 | 7-9 | 41 |
| F124N | I229 | 8-10 | 21 |
| F124T | I267 | 7-9 | 56 |
| F124T | I329 | 7-8 | 87 |
| F124H | I336 | 7-9 | 41 |
| F124H | I388 | 7-9 | 41 |
| F124K | I399 | 8-10 | 23 |
| F124G | I453 | 7 | 49 |
| F124Y | I472 | 5-7 | 66 |
| F124W | I505 | 5-7 | 56 |
| F124A | I104 | 7-9 | 32 |
| F124V | I115 | 7-9 | 53 |
| N71S F124K | 139CB1 | 9 | 16 |
| Y68G F124Q | 139DC1 | 4 | 143 |
| Y68G F124W | 139EC8 | 5-7 | 69 |

### EXAMPLE 2

### Adenoviral-Mediated Expression of NTR Mutants F124N and F124K/N71S Sensitises Cancer Cells to CB1954 to a Greater Extent than Expression of the WT Enzyme

In initiating this work, the assumption was made that improved versions of the E. coli NTR enzymes identified using a bacterial screening system would also activate CB1954 “more efficiently” than the WT enzyme in human cancer cells (so reducing the intratumoral CB1954 concentration and/or the duration of exposure of tumour cells to the drug required to generate sufficient activated prodrug for cell killing).

In this example we describe experiments that compare the efficiencies with which WT NTR and two mutant enzymes identified in the bacterial screen (F124N and F124K/N71S) sensitise a human cancer cell line (HeLa) to CB1954.

Methods

Virus Construction

NTR expression in HeLa cells was achieved by recombinant adenoviral mediated gene transfer. E1-deleted adenoviruses expressing the mutant enzymes were designed to be identical to the WT-expressing virus, “CTL102” (Djeha et al 2000) except for the respective coding change. The F124N coding sequence and 5′ flanking sequence was PCR amplified from the respective lambda phage using forward primer JG138A (5′-GCACGCTAGCAAGCTTCCACCATGGATATCATTTCTGTCGCC-3′) (SEQ ID NO:16) and reverse primer JG138B (5′-GCACAAGCTTGCTAGCTCATTACACTTCGGTTAAGGTGATG-3′) (SEQ ID NO:17). The product was cut with NheI and cloned into the XbaI site of pBluescript (Stratagene). A HindIII-BamHI fragment containing F124N was excised from the resultant plasmid and cloned into HindIII-BamHI digested pTX0374 (Djeha et al). A HindIII fragment containing the CMV promoter/enhancer was then cloned into the resultant vector. The Kozak consensus sequence present in the F124N (AAGCTT.CCA.CCATGg) (SEQ ID NO:18) differed from that present in the WT NTR expressing virus (AAGCTT.GCC.GCC.AGCCATGg) (SEQ ID NO:19). It was therefore removed by Ncol digestion and replaced with the equivalent Ncol fragment from pTX0374 (a plasmid containing wild type NTR used to construct CTL102). The CMV.F124N fragment was then cut out using SmaI and NheI, blunted and cloned into PmeI-digested vector pTX0398 (the transfer vector pPS1128 described in Djeha et al2000 but containing a PmeI site).

The F124K/N71S coding sequence and 5′ flanking sequence were PCR amplified from the respective lambda phage using primers SC1 (5′-AGTCCAAGCTTGCCGCCAGCCATGGATATCATTTCTGTCGCCTTAAAGCG-3′) (SEQ ID NO:20) and SC2 (5′-TGAGGATCCTTACACTTCGGTTAAGGTGATGTTTTGC-3′) (SEQ ID NO:21) which (i) introduced a unique HindIII site at the start of the NTR coding sequence and (ii) incorporated the CTL102 Kozak sequence. A BamHI site was introduced at the 3′ end of NTR to enable F124K/N71S to be cloned into HindIII-BamHI-cut pTX0374 as a HindIII-BamHI fragment. A HindIII fragment containing the CMV promoter/enhancer was then cloned into this vector. The CMV.F124KN71S fragment was then cut out using SpeI and cloned into SpeI digested pPS1128. Recombinant adenoviruses expressing respectively NTR F124N (“CTL802”) and F124K/N71S (“CTL805”) were rescued by homologous recombination in PerC6 cells and purified stocks prepared and titred as described for CTL102 (Djeha et al 2000).

CB1954 Sensitisation Experiments

Sensitisation of HeLa cells to CB1954 was assayed using the following protocol. Cells were infected with NTR-expressing viruses in suspension (2 hours) at a range of MOIs prior to plating into microtitre plates (10⁴ cells/well). After a 24 hour expression period, CB1954 was applied at a range of concentrations (0-50 μM) and after a 5 hour exposure to the prodrug, cell viability was assessed using the Promega MTS cell substrate killing assay (2-3 hour incubation before plate reading at OD450 nm). Under these conditions, for a given MOI and [CB1954], expression of both F124N and F124KN71S was consistently found to result in more extensive cell killing than that caused by expression of the WT enzyme. Adenovirus titreing by plaque formation on helper cells is however an error-prone process. To correct for this, experiments were performed with multiple independent titred preparations of each virus.

Results

FIG. 10 A, B, and C shows the results of an experiment in which the viruses used comprised a mixture of three preparations of each NTR-expressing virus (1:1:1). The titres of these mixes were assumed to be the means of the respective experimentally determined titres. For western blot analysis of NTR expression for normalisation purposes, whole cell extracts were resolved by SDS-PAGE on an 11% separation gel and blotted onto a nitrocellulose membrane. NTR was detected using a sheep anti-NTR serum (1:1000 diluted), donkey anti-sheep IgG labelled with HRP (horseradish peroxidase) and SuperSignal West Pico Chemiluminescence substrate (Pierce), analysed with an Alpha Innotech Imager Model #2.3.1. The relative loading of each well was determined by Coomassie blue staining of the gel post transfer.

As shown, at almost all MOIs and CB1954 concentrations used, CTL802 mediated greater sensitisation to CB1954 killing than CTL102. CTL805 mediated a greater effect still. Although in this experiment the improved killing achieved with F124N was moderate, the western blot in FIG. 11A shows that the level of F124N expression was lower than in WT NTR-expressing cells. This provides support for F124N possessing an improved capacity to activate CB1954 in cancer cells but possibly points to a reduced stability compared to WT. The killing due to F124KN71S expression was more marked. In this case however the level of enzyme expression was more similar to that of WT. Overall the data are consistent with the double mutant enzyme possessing more CB1954-activating activity than the WT enzyme.

In conclusion this experiment provides evidence that the F124N and F124K/N71S NTR mutants isolated using the bacterial screen can sensitise a human cancer cell line to CB1954 more effectively than the WT E.coli enzyme.

### EXAMPLE 3

### Kinetic Characterisation of Mutant NTRs

The observation that expression of certain NTR mutants increased the sensitization of E. coli to CB1954 beyond that observed with the WT enzyme was consistent with the mutant enzymes possessing increased catalytic activity. This was examined by kinetic analysis of selected mutants in vitro.

Wild type NTR and selected mutants were purified as described by Lovering et al., 2001. Steady state kinetic studies were carried out by monitoring the disappearance of nitrofurazone ( [symbol] =12,960, Zenno, et al., 1994) and nitrofurantoin ( [symbol] =12,020, McOsker, et al., 1992) at 420 nm or the disappearance of reduced benzoquinone ( [symbol] =18,5000), 2-nitrofuran ( [symbol] =10,250, McOsker, et al., 1992), 2-nitrobenzamide ( [symbol] =9,750, McOsker, et al., 1992) and 4 nitrobenzamide ( [symbol] =9,720, McOsker, et al., 1992) at 300 nm. The formation of the 4 hydroxylamine product of CB1954 reduction was monitored at 420 nm ( [symbol] =7900, Richard Knox, personal communication).

All reactions were performed in quartz cuvettes with either a 0.1-, 0.5-, or 1-cm pathlength. In all cases the reaction was initiated by the addition of a small amount of cold enzyme solution to the reaction mix. Assays were performed in 10 mM Tris HCL pH 7.0. The temperature of each reaction was maintained at 25° C. by means of a circulating water bath. All substrates examined were dissolved in DMSO, with the final concentration of organic solvent not exceeding 4% (v/v), concentrations>5% (v/v) give definable enzyme inhibition. In all assay the final DMSO concentration was at 4%. All steady state data were collected in an aerobic environment. Kinetic data were collected at concentration ranges extending from 0.1 of Kₘ to the maximum possible concentration permitted by substrate solubility or optical absorbance. In all cases maximum substrate concentrations exceeded 5×Kₘ. All data was analysed using the commercial package Sigma Plot™ and fit with non-linear regression to a rectangular hyperbola of the form:
y=ax/b+x

The results shown in FIG. 12A, B and C and Table 4 show that all mutants analysed showed an improvement in either Kₘ for CB1954 or k_{cat}. None displayed an improvement in both parameters. The mutant displaying the best bimolecular rate constant vs. the second substrate was T41L. F124H and F124K both showed significant improvement in k_{cat}/Kₘ for both nucleotide and second substrate. Y68G displayed a large improvement in catalytic activity vs. second substrate but not in k_{cat}/Kₘ as this was offset by an increased Km for CB1954. Overall these data provide evidence that improved catalytic activity underlies the improved efficiency of sensitization of E.coli to CB1954 with respect to the WT NTR enzyme.

TABLE 4
Kinetic parameters of a series of selected NTR mutants

| Enzyme | Fixed substrate | Variable substrate | Kₘ (μM) | k_{cat} (min⁻¹) | k_{cat}/Kₘ |
|---|---|---|---|---|---|
| Wild type | Nitrofurazone | NADH | 7 ± 1 | 657 ± 23 | 98 ± 18 |
| | NADH | CB1954 | 852 ± 8 | 342 ± 25 | 0.4 ± 0.1 |
| | NADH | Nitrofurazone | 157 ± 4 | 683 ± 3 | 4 ± 1 |
| F124H | Nitrofurazone | NADH | 3 ± 0.4 | 619 ± 13 | 193 ± 33 |
| | NADH | CB1954 | 526 ± 10 | 356 ± 35 | 0.7 ± 0.2 |
| | NADH | Nitrofurazone | 104 ± 10 | 643 ± 16 | 6 ± 2 |
| F124K | Nitrofurazone | NADH | 1 ± 0.2 | 723 ± 9 | 516 ± 43 |
| | NADH | CB1954 | 371 ± 12 | 343 ± 43 | 0.9 ± 0.1 |
| | NADH | Nitrofurazone | 53 ± 5 | 758 ± 15 | 14 ± 3 |
| T41L | Nitrofurazone | NADH | 5 ± 2 | 2111 ± 35 | 430 ± 20 |
| | NADH | CB1954 | 871 ± 77 | 972 ± 82 | 1.1 ± 0.2 |
| | NADH | Nitrofurazone | 79 ± 11 | 2108.4 ± 36.7 | 27 ± 3 |
| Y68G | Nitrofurazone | NADH | 22 ± 1 | 3286 ± 33 | 146 ± 23 |
| | NADH | CB1954 | 1841 ± 44 | 690 ± 54 | 0.4 ± 0.1 |
| | NADH | Nitrofurazone | 699 ± 24 | 3541 ± 24 | 5 ± 1 |

References

1. Djeha A H, Hulme A, Dexter M T, Mountain A, Young L S, Searle P F, Kerr D J, Wrighton C J (2000). Expression of Escherichia coli B nitroreductase in established human tumor xenografts in mice results in potent antitumoral and bystander effects upon systemic administration of the prodrug CB1954. Canver Gene Therapy 7: 721-731.

2. Friedlos F, Quinn J, Knox R J and Roberts J J (1992). The properties of total adducts and interstrand crosslinks in the DNA of cells treated with CB 1954. Exceptional frequency and stability of the crosslink. Biochem Pharmacol 43: 1249-1254.

3. Grove J I, Searle, P F, Weedon, S J, Green N K, McNeish I A and Kerr D J (1999). Virus-directed enzyme prodrug therapy using CB1954. Anti-Cancer Drug Design 14: 461-472.

4. Knox R J, Friedlos F, Jarman M and Roberts J J (1988). A new cytotoxic, DNA interstrand crosslinking agent, 5-(aziridin-1-yl)-4-hydroxylamino-2-nitrobenzamide, is formed from 5-(aziridin-1-yl)-2,4-dinitrobenzamide (CB1954) by a nitroreductase enzyme in Walker carcinoma cells. Biochem Pharmacol 37: 4661-4669.

5. Knox R J, Friedlos F, Marchbank T and Roberts J J (1991). Bioactivation of CB 1954: reaction of the active 4-hydroxylamino derivative with thioesters to form the ultimate DNA-DNA interstrand crosslinking species. Biochem Pharmacol 42: 1691-1697.

6. Lovering A L, Hyde E I, Searle P F and White S A (2001). The structure of Escherichia coli nitroreductase complexed with nicotinic acid: three crystal forms at 1.7 Å, 1.8 Å, and 2.4 Å resolution. J Mol Biol 309: 203-213.

7. McNeish I A, Searle P F, Young L S and Kerr D J (1997). Gene-directed enzyme prodrug therapy for cancer. Advanced Drug Delivery Reviews 26: 173-184.

8. McOsker C C and Fitzpatrick P M (1994). Nitrfurantoin: mechanism of action and implications for resistance development in common uropathogens. J Antimicrob Chemother 33 Suppl A:23-30.

9. Parkinson G N, Skelly J V and Neidle S (2000). Crystal structure of FMN-dependent nitroreductase from Escherichia coli B: a prodrug-activating enzyme. J Med Chem 43: 3624-3631.

10. Zenno S, Koike H, Tanokura M and Saigo K (1996). Conversion of NfsB, a minor Escherichia coli nitroreductase, to a flavin reductase similar in biochemical properties to FRase I, the major flavin reductase in Vibrio fischeri, by a single amino acid substitution. J Bacteriology 178: 4731-4733.

All references cited herein are hereby incorporated by reference in their entireties.

### OTHER EMBODIMENTS

Other embodiments will be evident to those of skill in the art. It should be understodd that the foregoing detailed description is provided for clarity only and is merely exemplary. The spirit and scope of the present invention are not limited to the above examples, but are encompassed by the following claims.

1. A recombinant mutant nitroreductase encoded by a mutated equivalent of the E.coli NFSB gene, characterised in that it has an increased nitroreductase activity for CB1954 compared to the wild-type enzyme.

2. A nitroreductase according to claim 1, characterised in that said nitroreductase comprises a substitution of serine 40 with an amino acid selected from a group consisting of alanine, glycine and threonine.

3. A nitroreductase according to claim 1, characterised in that said nitroreductase corresponds to the wild-type sequence of FIG. 9 (SEQ ID NO: 1), wherein serine 40 is substituted by an amino acid selected from the group consisting of alanine, glycine and threonine, and optionally also having substitutions, insertions or deletions at residues other than serine 40.

4. A nitroreductase according to claim 1, characterised in that said nitroreductase comprises a substitution of threonine 41 with an amino acid selected from a group consisting of asparagine, glycine, isoleucine, leucine and serine.

5. A nitroreductase according to claim 1, characterised in that said nitroreductase corresponds to the wild-type sequence of FIG. 9 (SEQ ID NO: 1), wherein threonine 41 is substituted by an amino acid selected from the group consisting of asparagine, glycine, isoleucine, leucine and serine, and optionally also having substitutions, insertions or deletions at residues other than threonine 41.

6. A nitroreductase according to claim 1, characterised in that said nitroreductase comprises a substitution of tyrosine 68 with an amino acid selected from a group consisting of alanine, asparagine, aspartate, cysteine, glutamine, glycine, histidine, serine and tryptophan.

7. A nitroreductase according to claim 1, characterised in that said nitroreductase corresponds to the wild-type sequence of FIG. 9 (SEQ ID NO:1), wherein tyrosine 68 is substituted by an amino acid selected from the group consisting of alanine, asparagine, aspartate, cysteine, glutamine, glycine, histidine, serine and tryptophan, and optionally also having substitutions, insertions or deletions at residues other than tyrosine 68.

8. The nitroreductase of claim 7, characterised in that said nitroreductase is a double mutant comprising a first substitution of tyrosine 68 to glycine (Y68G) and a second substitution of phenylalanine 124 to tryptophan (F124W).

9. A nitroreductase according to claim 1, characterised in that said nitroreductase comprises a substitution of phenylalanine 70 with an amino acid selected from a group consisting of alanine, cysteine, glutamine, glutamate, glycine, isoleucine, leucine, proline, serine, threonine and valine.

10. A nitroreductase according to claim 1, characterised in that said nitroreductase corresponds to the wild-type sequence of FIG. 9 (SEQ ID NO:1), wherein phenylalanine 70 is substituted by an amino acid selected from the group consisting of alanine, cysteine, glutamine, glutamate, glycine, isoleucine, leucine, proline, serine, threonine and valine, and optionally also having substitutions, insertions or deletions at residues other than phenylalanine 70.

11. A nitroreductase according to claim 1, characterised in that said nitroreductase comprises a substitution of asparagine 71 with an amino acid selected from a group consisting of aspartate, glutamine and serine.

12. A nitroreductase according to claim 1, characterised in that said nitroreductase corresponds to the wild-type sequence of FIG. 9 (SEQ ID NO: 1), wherein asparagine 71 is substituted by an amino acid selected from the group consisting of aspartate, glutamine and serine, and optionally also having substitutions, insertions or deletions at residues other than asparagine 71.

13. The nitroreductase of claim 12 characterised in that said nitroreductase is a double mutant comprising a first substitution of asparagine 71 to serine (N71S) and a second substitution of phenylalanine 124 to lysine (F124K).

14. A nitroreductase according to claim 1 characterised in that said nitroreductase comprises a substitution of glycine 120 with an amino acid selected from a group consisting of alanine, serine and threonine.

15. A nitroreductase according to claim 1, characterised in that said nitroreductase corresponds to the wild-type sequence of FIG. 9 (SEQ ID NO:1), wherein glycine 120 is substituted with an amino acid selected from a group consisting of alanine, serine and threonine.

16. A nitroreductase according to claim 1, characterised in that said nitroreductase comprises a substitution of phenylalanine 124 with an amino acid selected from a group consisting of asparagines, cysteine, glycine, lysine, methionine, tryptophan and tyrosine.

17. A nitroreductase according to claim 1, characterised in that said nitroreductase corresponds to the wild-type sequence of FIG. 9 (SEQ ID NO:1), wherein phenylalanine 124 is substituted by an amino acid selected from the group consisting of asparagine, cysteine, glycine, lysine, methionine, tryptophan and tyrosine, and optionally also having substitutions, insertions or deletions at residues other than phenylalanine 124.

18. An isolated polynucleotide encoding a nitroreductase according to claim 1.

19-24. (canceled)

25. A recombinant mutant nitroreductase encoded by a mutated E.coli NfsB gene, characterised in that it has an increased nitroreductase activity compared to the wild-type enzyme and comprises the substitution of phenylalanine 124 with an amino acid selected from the group consisting of alanine, glutamine, histidine, isoleucine, leucine, serine, threonine or valine.

26. A recombinant E.coli NfsB nitroreductase mutant corresponding to the wild-type sequence of FIG. 9 (SEQ ID NO: 1), characterised in that phenylalanine 124 is substituted by an amino acid selected from the group consisting of alanine, glutamine, histidine, isoleucine, leucine, serine, threonine or valine, having nitroreductase activity greater than that of the wild-type protein, and optionally also having substitutions, insertions or deletions at residues other than phenylalanine 124.

27. An isolated polynucleotide encoding a nitroreductase according to claim 25.

28-34. (canceled)

35. A vector comprising an isolated polynucleotide according to claim 18.

36. A vector according to claim 35 characterised in that said vector provides tissue-specific expression of the encoded nitroreductase.

37. A vector according to claim 36 characterised in that said vector comprises a TCF-responsive element operably linked to said polynucleotide.

38. A vector according to any of claims 35 to 37 characterised in that said vector is a virus.

39. A vector according to claim 38 characterised in that said viral vector is an adenovirus.

40. (canceled)

41. A host cell comprising an isolated polynucleotide according to of either of claims 18 or 27 or a vector according to any one of claims 35 to 39.

42-44. (canceled)

45. A method of treating cancer in a mammalian subject, comprising administering the isolated polynucleotide of either of claims 18 or 27, or the vector according to any one of claims 35 to 39, allowing a suitable time for expression of the encoded nitroreductase to occur, and administering a prodrug capable of being activated by said expressed nitroreductase.

46. A recombinant mutant nitroreductase with increased nitroreductase activity as compared to the wild-type enzyme, characterised in that said nitroreductase is encoded by a mutated Salmonella NFSB gene.

47. A recombinant mutant nitroreductase with increased nitroreductase activity as compared to the wild-type enzyme, characterised in that said nitroreductase is encoded by a mutated Enterobacter NFSB gene.

48. A nitroreductase according to either of claims 46 or 47 characterised in that it has an increased nitroreductase activity for prodrugs.

49. A nitroreductase according to claim 48, characterised in that it has an increased nitroreductase activity for nitrobenzamide prodrugs.

50. A nitroreductase according to claim 49, characterised in that it has an increased nitroreductase activity for CB1954.

51. A method for treating cancer comprising administering a nitroreductase according to any one of claims 1-17 to a mammalian subject in the presence of a prodrug capable of being activated by said nitroreductase.

52. A method for converting a prodrug into a cytotoxic agent comprising administering a nitroreductase according to any one of claims 1-17 or an isolated polynucleotide according to claim 18 in the presence of said prodrug.

53. A method for converting a nitrobenzamide prodrug into a cytotoxic agent comprising administering a nitroreductase according to any one of claims 1-17 or an isolated polynucleotide according to claim 18 in the presence of said prodrug.

54. A method for converting CB1954 into a cytotoxic agent comprising administering a nitroreductase according to any one of claims 1-17 or an isolated polynucleotide according to claim 18 in the presence of CB1954.

55. A method for treating cancer by converting a prodrug into a cytotoxic agent comprising administering a nitroreductase according to any one of claims 1-17 or an isolated polynucleotide according to claim 18 in the presence of said prodrug.

56. A method for treating cancer comprising administering a nitroreductase according to claims 25 or 26 to a mammalian subject in the presence of a prodrug capable of being activated by said nitroreductase.

57. A method for converting a prodrug into a cytotoxic agent comprising administering a nitroreductase according to claims 25 or 26 or an isolated polynucleotide according to claim 27 in the presence of said prodrug.

58. A method for converting a nitrobenzamide prodrug into a cytotoxic agent comprising administering a nitroreductase according to claims 25 or 26 or an isolated polynucleotide according to claim 27 in the presence of said prodrug.

59. A method for converting CB1954 into a cytotoxic agent comprising administering a nitroreductase according to claims 25 or 26 or an isolated polynucleotide according to claim 27 in the presence of CB1954.

60. A method for treating cancer by converting a prodrug into a cytotoxic agent comprising administering a nitroreductase according to claims 25 or 26 or an isolated polynucleotide according to claim 27 in the presence of said prodrug.

61. A pharmaceutical composition comprising the nitroreductase according to any one of claims 1, 25, or 26 or of an isolated polynucleotide according to either of claims 18 or 27, in a pharmaceutically acceptable diluent or excipient.

62. A pharmaceutical composition comprising the vector of any one of claims 35 to 37 in a pharmaceutically acceptable diluent or excipient.

63. A pharmaceutical composition comprising the host cell of claim 41 in a pharmaceutically acceptable diluent or excipient.

64. A method for the design of, or screening for, improved prodrugs comprising contacting a test prodrug with the nitroreductase according to any one of claims 1 to 17, 25, or 26, and screening for cytotoxic activity.
